(19) United States
(12) Patent Application Publication
(10) Pub. No.: US 2011/0084001 A1
(43) Pub. Date: Apr. 14, 2011

(54) METHOD OF OLIGOMERIZATION OF AN OLEFINIC HYDROCARBON FEED USING A CATALYST BASED ON A MACROPOROUS SILICA-ALUMINA

(75) Inventors: Amandine Cabiac, Lyon (FR); Alexandra Chaumonnot, Lyon (FR)

(73) Assignee: IFP Energies nouvelles, Rueil-Malmaison Cedex (FR)

(21) Appl. No.: 12/900,087

(22) Filed: Oct. 7, 2010

(30) Foreign Application Priority Data
Oct. 8, 2009 (FR) 09/04.818

(51) Int. Cl. C07C 2/12; C10G 50/00

(52) U.S. Cl. 208/135; 585/533

(57) ABSTRACT

A method of oligomerization of an olefinic hydrocarbon feed is described, consisting of contacting said feed with at least one catalyst comprising at least one silica-alumina, the silica content by weight of said catalyst being between 5 and 95 wt. % and the porosity of said silica-alumina when formed being such that:
i) the volume V1 of mesopores with a diameter comprised between 4 and 15 nm represents 30-80% of the total pore volume measured with a mercury intrusion porosimeter,
ii) the volume V2 of macropores with a diameter greater than 50 nm represents from 15 to 80% of the total pore volume measured with a mercury intrusion porosimeter

### FIELD OF THE INVENTION

The present invention relates to any method of oligomerization of olefins for the production of fuels, for example the production of gasoline and/or kerosene and/or diesel fuel, from light olefinic feeds containing between 2 and 10 carbon atoms per molecule using an oligomerization catalyst that comprises at least one silica-alumina having a specified pore distribution when it is formed, the silica content by weight of said catalyst being between 5 and 95 wt. %.

### PRIOR ART

The methods of oligomerization of light olefins intended for the production of olefins of higher molecular weight are widely used in the field of refining and petrochemistry, with the aim of upgrading the light olefins to bases for fuels, of the gasoline, kerosene or diesel fuel type, or to solvents. The oligomerization reactions are carried out in the presence of a catalyst, most often a solid catalyst. The olefins combine to form dimers, trimers, tetramers, etc., the degree of polymerization of the olefins depending on the type of catalyst used and the operating conditions of temperature and pressure used. The advantage of the oligomerization method, relative to other methods in the field of refining and petrochemistry that lead to the same product range and are well known to a person skilled in the art, resides in the fact that the products thus obtained are sulphur-free and have a very low content of aromatic compounds. The solid oligomerization catalysts often mentioned in the literature are acid catalysts, the main examples of which, in the area of oligomerization of light olefins, are catalysts of the impregnated phosphoric acid type on a solid support (for example U.S. Pat. No. 2,913,506 and U.S. Pat. No. 3,661,801), silica-aluminas (for example patents U.S. Pat. No. 4,197,185, US 4,544,791 and EP 0,463,673), zeolites (for example patents U.S. Pat. No. 4,642,404 and U.S. Pat. No. 5,284,989) and, to a lesser extent, heteropolyanions (for example patent IN 170,903).

The catalysts of the impregnated phosphoric acid type on a solid support (SPA) have a good oligomerization activity as well as a high yield of products that can be upgraded to the gasoline fraction. These catalysts are, however, difficult to handle, in particular at the moment of discharge from the unit associated with the method, because of their tendency to increase in weight in the presence of olefins. Moreover, said catalysts of the impregnated phosphoric acid type on a solid support are degraded in the course of the reaction and cannot be regenerated.

Zeolites are acidic materials that are active for the oligomerization reaction of light olefins owing to the nature of the sites involved. These catalysts are therefore used for said applications. An appropriate choice of zeolite catalyst permits, through suitable geometric selectivity, increased production of oligomers that are less branched than when an amorphous catalyst is used. Careful selection of the type of zeolite as oligomerization catalyst therefore makes it possible to modulate the selectivity of the reaction and can therefore result in oligomers having a degree of branching less than that of oligomers resulting from reactions catalysed by catalysts not requiring any selectivity of shape. This gain in selectivity is favourable in the context of the production of diesel fuel of good quality, i.e. with high cetane number, but rather unfavourable for example for the production of gasoline having a good octane number. Catalysts of the heteropolyanion type are used for the oligomerization reaction of light olefins. These catalysts are not thermally stable and therefore lead to low conversions and oligomers with a degree of polymerization that is limited owing to the restricted operating temperature.

The general term silica-alumina covers a wide range of amorphous aluminosilicate catalysts having textural and physicochemical properties that are suited to the oligomerization reaction. The texture and acidity properties of the material, dictated by the method of preparation of the catalyst and well known to a person skilled in the art, determine the activity and selectivity of the catalyst. It is known that catalysts based on amorphous silica-alumina having a large pore volume impose fewer geometric constraints than their zeolite homologues and are therefore interesting candidates for the production of gasoline and/or kerosene of good quality through the oligomerization reaction of light olefins. For example, the catalysts disclosed in patent EP 0,463,673 for the oligomerization of propylene to products that can be upgraded in the gasoline and/or kerosene pool, are amorphous silica-aluminas characterized by large specific surfaces, between 500 and 1000 m²/g.

One way of evaluating the performance of an oligomerization catalyst consists of estimating the selectivity of said catalyst for the required reaction products, namely oligomers having a boiling point less than 225° C.

The selectivity by mass of a catalyst for a product P under given operating conditions is defined as the ratio of the mass of the product P to the sum of the masses of the reaction products. The selectivity for the product P increases as the secondary reactions, defined as reactions leading to the formation of products different from the required product, are minimized. In the case of the oligomerization of light olefins, subsequent oligomerization reactions or uncontrolled oligomerization reactions lead to the production of products having a molecular weight greater than the molecular weight of the required products. On the other hand, cracking reactions lead to the production of products having a molecular weight less than the molecular weight of the required products. These two types of reactions therefore have to be minimized in order to improve the selectivity for a product or a family of products. One way of minimizing these secondary reactions is to limit problems of diffusion within the catalyst bed.

### SUMMARY AND BENEFIT OF THE INVENTION

The present invention relates to a method of oligomerization of an olefinic hydrocarbon feed consisting of contacting said feed with at least one catalyst comprising at least one silica-alumina, the silica content by weight of said catalyst being between 5 and 95 wt. % and the porosity of said silica-alumina when formed being such that:
i) the volume V1 of mesopores having a diameter between 4 and 15 nm represents 30-80% of the total pore volume measured with a mercury intrusion porosimeter,
ii) the volume V2 of macropores having a diameter greater than 50 nm represents from 15 to 80% of the total pore volume measured with a mercury intrusion porosimeter.

Preferably, the oligomerization catalyst is constituted entirely by said silica-alumina and is in the form of extrudates.

It was discovered, surprisingly, that a catalyst comprising at least one silica-alumina having a specified pore distribution when it is formed, in particular a high macropore volume, i.e. representing from 15 to 80% of the total pore volume, leads to improved catalyst performance in terms of selectivity for the desired products when it is used in a method of oligomerization of an olefinic hydrocarbon feed containing olefinic molecules having from 2 to 10 carbon atoms per molecule, said method permitting the production of fuel, for example the production of gasoline and/or kerosene and/or diesel fuel. In fact, the particular physicochemical properties combined with suitable textural properties, in particular the properties associated with macroporosity, of the oligomerization catalyst used in the method of said invention lead to a reduction of the secondary reactions described above and therefore to an improvement of the selectivity for the required products during application of said catalyst in a method of oligomerization of an olefinic hydrocarbon feed containing olefinic molecules having from 2 to 10 carbon atoms per molecule. Thus, the improvement in intraparticle diffusion of the reagents and the products within at least the silica-alumina present in the catalyst used in the oligomerization method of the invention is reflected in better selectivity for the required oligomers, which have a boiling point generally between 50 and 225° C. More precisely, the oligomerization catalyst used in the method of the invention is more selective not only for products having a boiling point less than 155° C., corresponding to the products that can be incorporated in a gasoline fraction, but also for products having a boiling point between 155 and 225° C., corresponding to the products that can be incorporated in a kerosene fraction. The oligomerization catalyst used in the method according to the invention promotes the production of oligomers that can easily be incorporated in a gasoline and/or kerosene and/or diesel fuel fraction at the expense of the production of heavier products, which cannot be upgraded directly in the desired gasoline, kerosene and diesel fuel fractions.

Characterization Techniques

The catalyst based on at least one silica-alumina, used in the oligomerization method of the invention, is characterized by several analysis methods and in particular by wide-angle X-ray diffraction (XRD), by nitrogen adsorption isotherm, by mercury intrusion porosimetry, by transmission electron microscopy (TEM) optionally combined with energy-dispersion X-ray analysis (EDX), by nuclear magnetic resonance of the solid of the aluminium atom (²⁷Al MAS NMR), by infrared (IR) and X-ray fluorescence (XRF) or Atomic Absorption (AA) spectroscopy. The density of the catalyst used in the method of the invention is also evaluated.

The technique of wide-angle X-ray diffraction (values of angle 2θ comprised between 5° and 70°) makes it possible to characterize a crystalline solid defined by the repetition of a structural unit or unit cell at the molecular scale. In the following disclosure, X-ray analysis is performed on powder with a diffractometer operating in reflection and equipped with a rear monochromator using the radiation from copper (wavelength of 1.5406 Å). The peaks usually observed in the diffraction patterns corresponding to a given value of angle 20 are associated with the interplanar spacings d₍ₕₖₗ₎ characteristic of the structural symmetry or symmetries of the catalyst ((hkl) being the Miller indices of the reciprocal lattice) by the Bragg relation: 2 d₍ₕₖₗ₎*sin(θ)=n*λ. This indexation then makes it possible to determine the lattice parameters (abc) of the direct lattice. As an example and advantageously within the scope of the invention, the two most intense peaks present in the diffraction pattern of the oligomerization catalyst used for application of the method of the invention are located in a position corresponding to a d comprised between 139 Å and 1.40 Å and a d comprised between 1.97 Å and 2.00 Å. These peaks are associated with the presence of gamma alumina in the silica-alumina contained in the oligomerization catalyst. By gamma alumina is meant, hereinafter, among other things and for example, an alumina included in the group comprising the following aluminas: gamma cubic, gamma pseudo-cubic, gamma tetragonal, gamma poorly or slightly crystallized, gamma with large surface area, gamma with small surface area, gamma derived from bulk boehmite, gamma derived from crystallized boehmite, gamma derived from slightly or poorly crystallized boehmite, gamma derived from a mixture of crystallized boehmite and an amorphous gel, gamma derived from an amorphous gel, gamma evolving towards delta. For the positions of the diffraction peaks of the eta, delta and theta aluminas, reference may be made to the article by B. C. Lippens and J. J. Steggerda, in “Physical and Chemical Aspects of Adsorbents and Catalysts”, E. G. Linsen (Ed.), Academic Press, London, 1970, 171. For the catalyst used in the method according to the invention, the X-ray diffraction pattern shows a broad peak characteristic of the presence of amorphous silica. Moreover, throughout the text that follows, the alumina fraction of the oligomerization catalyst can contain an amorphous fraction that is difficultly detectable by XRD techniques. It will therefore be implied hereinafter that the alumina fraction can contain an amorphous or poorly crystallized fraction.

Nitrogen adsorption isotherm analysis corresponding to the physical adsorption of nitrogen molecules in the catalyst porosity by a progressive increase in pressure at constant temperature provides information on the particular textural characteristics (pore diameters, type of porosity, specific surface) of the oligomerization catalyst used in the method according to the invention. In particular, it enables us to find the specific surface and the mesopore distribution of said catalyst. By specific surface is meant the BET specific surface (S_{BET} in m²/g) determined by nitrogen adsorption according to standard ASTM D 3663-78 established using the BRUNAUER-EMMETT-TELLER method described in the periodical “The Journal of American Society”, 1938, 60, 309. The pore distribution representative of a population of mesopores centred in a range from 1.5 nm to 50 nm is determined by the Barrett-Joyner-Halenda (BJH) model. The nitrogen adsorption/desorption isotherm according to the BJH model is described in the periodical “The Journal of American Society”, 1951, 73, 373, written by E. P. Barrett, L. G. Joyner and P. P. Halenda. In the following account, the “nitrogen adsorption volume of the catalyst” corresponds to the volume measured for P/P₀=0.99, a pressure for which it is assumed that the nitrogen has filled all the pores.

In the following disclosure, the “mercury volume of the catalyst” corresponds to the volume measured with a mercury intrusion porosimeter according to standard ASTM D4284-83 at a maximum pressure of 4000 bar, using a surface tension of 484 dyne/cm and a contact angle for the oligomerization catalyst comprising at least one amorphous silica-alumina of 140°. The mean mercury diameter is defined as being a diameter such that all the pores of size less than this diameter constitute 50% of the pore volume (V_{Hg}), in a range between 3.6 nm and 100 nm. The wetting angle was taken as 140° following the recommendations of the work “Techniques de l'ingenieur, traité analyse et caractérisation”, 1050, by J. Charpin and B. Rasneur. For greater accuracy, the value of the mercury volume in ml/g given hereinafter corresponds to the value of the total mercury volume in ml/g measured on the sample minus the value of the mercury volume in ml/g measured on the same sample for a pressure corresponding to 30 psi (about 2 bar or 0.2 MPa). For better characterization of the pore distribution, the following criteria of pore distribution in mercury are defined: the volume V1 which is the volume corresponding to the pores having a diameter in the range from 4 nm to 15 nm, the volume V2 which is the volume of macropores having a diameter greater than 50 nm and the volume V3 which is the volume of pores having a diameter greater than 25 nm.

Analysis by transmission electron microscopy (TEM) is a technique that is also widely used for characterizing the oligomerization catalyst comprising at least one silica-alumina used in the oligomerization method according to the invention. The latter permits the formation of an image of the solid wider investigation, the contrasts observed being characteristic of the structural organization, texture, morphology or composition of the particles observed, the resolution of the technique reaching a maximum of 0.2 nm. For this, an electron microscope (of the Jeol 2010 or Philips Technai20F type optionally with scanning) equipped with an energy-dispersive X-ray (EDX) spectrometer (for example a Tracor or an Edax) is used. The EDX detector must permit detection of the light elements. The combination of these two tools, TEM and EDX, makes it possible to combine imaging and local chemical analysis with good spatial resolution. For this type of analysis, the samples are finely ground, dry, in a mortar. The powder is then embedded in resin for making ultrafine sections with a thickness of about 70 nm. These sections are collected on copper gratings covered with a film of amorphous carbon with holes, serving as support. They are then introduced into the microscope for observation and analysis under high vacuum. Under imaging, the sample zones of the resin zones are easily distinguished. A certain number of analyses are then performed, 10 as a minimum, preferably between 15 and 30, on different zones of the sample. The size of the electron beam for analysis of the zones (approximately determining the size of the zones analysed) is 50 nm in diameter at most, preferably 20 nm, even more preferably 10, 5, 2 or 1 nm in diameter. In scanning mode, the zone analysed will depend on the size of the zone scanned and not on the size of the generally reduced beam. Semi-quantitative processing of the X-ray spectra obtained using the EDX spectrometer gives the relative concentration of the elements Al and Si (in atom-%) and the atomic ratio Si/Al for each of the zones analysed. The mean value Si/Alₘ and the standard deviation σ of this set of measurements can then be calculated.

The oligomerization catalyst comprising at least one silica-alumina and used in the method of the invention was analysed by NMR MAS of the solid of ²⁷Al on a spectrometer from the Brüker company of type MSL 400, with a 4 ram probe. The speed of rotation of the samples is of the order of 11 kHz. Potentially, the NMR of aluminium makes it possible to distinguish three types of aluminium, with the chemical shifts stated below:
between 100 and 40 ppm, aluminium atoms of the tetracoordinated type, designated Al_{IV},
between 40 and 20 ppm, aluminium atoms of the pentaeoordinated type, designated Al_{V},
between 20 and −100 ppm, aluminium atoms of the hexacoordinated type, designated Al_{VI}.

The aluminium atom is a quadripolar nucleus. Under certain analysis conditions (low radiofrequency fields: 30 kHz, small pulse angle: π/2 and water-saturated sample), the technique of magic angle spinning (MAS) NMR is a quantitative technique. Breakdown of the NMR MAS spectra gives the quantity of the various species directly. The spectrum is adjusted in chemical shift relative to a 1M solution of aluminium nitrate. The aluminium signal is at zero ppm. It was decided to integrate the signals between 100 and 20 ppm for Al_{IV} and Al_{V}, which corresponds to area 1, and between 20 and −100 ppm for Al_{VI}, which corresponds to area 2. In the following disclosure of the invention, by proportion of octahedral Al_{VI} is meant the following ratio: area 2/(area 1+area 2).

The acidity of the oligomerization catalyst is measured by infrared spectroscopy. The IR spectra are recorded on a Nicolet interferometer of the Nexus-670 type at a resolution of 4 cm⁻¹ with apodization of the Happ-Gensel type. The sample (20 mg) is compressed into the form of a self-supported pellet and placed in an in-situ analysis cell (25 to 550° C., furnace offset from the IR beam, high vacuum of 10⁻⁶ mbar). The diameter of the pellet is 16 mm. The sample is pretreated in the following way in order to remove physically sorbed water and partially dehydroxylate the surface of the catalyst to give an image that is representative of the acidity of the catalyst in service:
temperature increase from 25 to 300° C. in 3 hours,
plateau of 10 hours at 300° C.,
temperature decrease from 300 to 25° C. in 3 hours.

The basic probe (pyridine) is then adsorbed at saturating pressure at 25° C. then thermodesorbed in the following stages:
25° C. for 2 hours under high vacuum,
100° C. for 1 hour under high vacuum,
200° C. for 1 hour under high vacuum,
300° C. for 1 hour under high vacuum.

A spectrum is recorded at 25° C. at the end of the pretreatment and in each desorption stage in transmission mode with an accumulation time of 100 s. The spectra are adjusted to isomass (20 mg exactly). The number of Lewis acid sites is proportional to the area of the peak the maximum of which is at around 1450 cm⁻¹, including any shoulder. The number of Bronsted acid sites is proportional to the area of the peak the maximum of which is at around 1545 cm⁻¹. The ratio of the number of Bronsted acid sites to the number of Lewis acid sites (B/L) is estimated as equal to the ratio of the areas of the two peaks described above. The area of the peaks at 25° C. is generally used. This ratio B/L is generally calculated from the spectrum recorded at 25° C. after adsorption of pyridine and the plateau of 2 h under high vacuum.

The overall composition of the oligomerization catalyst used in the method of the invention, and in particular the content of the element sodium, can be determined by X-ray fluorescence (XRF) on said catalyst in powder form or by Atomic Absorption (AA) after acid attack of said catalyst.

The tap density (TD) is measured as described in the work “Applied Heterogeneous Catalysis” by J. F. Le Page, J. Cosyns, P. Courty, E. Freund, J.-P. Franck, Y. Jacquin, B. Juguin, C. Marcilly, G. Martino, J. Miguel, R. Montarnal, A. Sugier, H. Van Landeghem, Technip, Paris, 1987, Chapter 6.2.4, 167. A graduated cylinder of a suitable size is filled with successive additions and, between two successive additions, the catalyst is packed by shaking the cylinder until a constant volume is reached. This measurement is generally carried out on 1000 cm³ of packed catalyst in a cylinder with a ratio of height to diameter of about 5:1. This measurement can preferably be carried out on automated equipment such as Autotap® marketed by Quantachrome®.

### DETAILED DESCRIPTION OF THE INVENTION

Hereinafter, by oligomerization is meant any reaction of addition of an olefin to another olefin.

The present invention relates to a method of oligomerization of an olefinic hydrocarbon feed consisting of contacting said feed with at least one catalyst comprising at least one silica-alumina, the silica content by weight of said catalyst being between 5 and 95 wt. % and the porosity of said silica-alumina when formed being such that:
i) the volume V1 of mesopores having a diameter between 4 and 15 nm represents from 30 to 80% of the total pore volume measured with a mercury intrusion porosimeter,
ii) the volume V2 of macropores having a diameter greater than 50 nm represents from 15 to 80% of the total pore volume measured with a mercury intrusion porosimeter.

Catalyst Used in the Oligomerization Method According to the Invention

The catalyst used in the oligomerization method according to the present invention is a non-zeolite catalyst comprising at least one silica-alumina, i.e. comprising silica and alumina.

According to the invention, the oligomerization catalyst has a silica content by weight between 5 and 95 wt. %, preferably between 18 and 60 wt. %, more preferably between 18 and 40 wt. % and even more preferably between 25 and 40 wt. %. Consequently, the Si/Al molar ratio of the oligomerization catalyst used in the method according to the invention is between 0.044 and 17, preferably between 0.185 and 1.27, more preferably between 0.185 and 0.56 and even more preferably between 0.28 and 0.56.

According to the invention, the silica-alumina once formed has a pore distribution such that:
i) the volume V1 of mesopores having a diameter between 4 and 15 nm represents from 30 to 80% of the total pore volume measured witph a mercury intrusion porosimeter,
ii) the volume V2 of macropores having a diameter greater than 50 nm represents from 15 to 80% of the total pore volume, preferably from 23 to 80% of said total pore volume and even more preferably from 35 to 80% of said total pore volume measured with a mercury intrusion porosimeter.

Advantageously, the silica-alumina once formed has a volume V3 of pores having a diameter greater than 25 nm comprised between 20 and 80% of the total pore volume, preferably between 25% and 60% of the total pore volume and even more preferably between 30 and 50% of the total pore volume, measured with a mercury intrusion porosimeter.

Said formed silica-alumina, contained in the catalyst used in the oligomerization method according to the invention, is a macroporous silica-alumina. The forming of said silica-alumina is described later on in the present description. Preferably, said silica-alumina is formed in the form of extrudates.

The formed silica-alumina has a total pore volume measured with a mercury intrusion porosimeter between 0.45 and 0.96 ml/g. The average diameter of the pores of the formed silica-alumina, obtained using the mercury intrusion porosimeter, is in a range from 2 to 15 nm, preferably in a range from 4 to 12 nm and even more preferably in a range from 5 to 12 nm. The formed silica-alumina has a total pore volume measured from the nitrogen adsorption isotherm comprised between 0.45 and 0.96 ml/g.

According to the invention, the silica-alumina present in the oligomerization catalyst has a content of cationic impurities preferably less than 0.1 wt. %, more preferably less than 0.05 wt. % and even more preferably less than 0.025 wt. %. By content of cationic impurities is meant the total content of alkalines, in particular sodium. Said silica-alumina has a content of anionic impurities preferably less than 1 wt. %, more preferably less than 0.5 wt. % and even more preferably less than 0.1. wt. %. The anionic impurities present in said oligomerization catalyst are in particular halides, in particular chlorides, as well as sulphates and nitrates. According to the invention, the formed silica-alumina, present in the oligomerization catalyst, has a BET specific surface between 100 and 550 m²/g, preferably between 150 and 500 m²/g, more preferably between 150 and 350 m²/g and even more preferably between 170 and 310 m²/g.

According to the invention, the X-ray diffraction pattern of the oligomerization catalyst advantageously contains at least the principal lines characteristic of at least one of the transition aluminas comprised in the group composed of the alpha, rho, chi, kappa, eta, gamma, theta and delta aluminas and preferably at least the principal lines characteristic of at least one of the transition aluminas comprised in the group composed of gamma, eta, theta and delta alumina, and more preferably at least the principal lines characteristic of gamma or eta alumina, and even more preferably the pattern contains the peaks at a d comprised between 1.39 and 1.40 Å and at a d comprised between 1.97 Å and 2.00 Å. The diffraction pattern of the oligomerization catalyst used in the method according to the invention, obtained by X-ray diffraction, corresponds to a mixture of silica and alumina with a certain variation between alumina, preferably gamma alumina, and silica depending on the SiO₂ content of the samples. More precisely, analysis by X-ray diffraction demonstrates the increase in amorphous character with respect to X-rays, which is accentuated with increasing silica content in comparison with alumina, preferably gamma alumina.

The tap density of the oligomerization catalyst is greater than 0.40 g/cm³, preferably greater than 0.45 g/cm³, and very preferably greater than 0.50 g/cm³.

The formed silica-alumina contained in the catalyst used in the method according to the invention is preferably a silica-alumina that is homogeneous at the micrometre scale, i.e. no phase separation can be distinguished, at the micrometre scale, between the alumina fraction and the silica fraction present in the silica-alumina. The formed silica-alumina present in the catalyst used in the method according to the invention is preferably a silica-alumina that is heterogeneous at the nanometre scale, i.e. phase separation is distinguished, at the nanometre scale, between the alumina fraction and the silica fraction present in the silica-alumina. The homogeneity at the micrometre scale and the heterogeneity at the nanometre scale are preferably analysed by scanning electron microscopy (SEM).

In one embodiment of the oligomerization catalyst used in the method of the invention, said catalyst contains at least two silica-alumina zones, said zones having Si/Al molar ratios less than or greater than the overall Si/Al molar ratio determined by X-ray fluorescence. Thus, a catalyst having an overall Si/Al molar ratio equal to 0.5 comprises for example two silica-alumina zones, one of the zones having an Si/Al molar ratio determined by TEM less than 0.5 and the other zone having an Si/Al molar ratio determined by TEM between 0.5 and 17.

In another embodiment of the oligomerization catalyst used in the method of the invention, said catalyst contains a single silica-alumina zone, said zone having an Si/Al molar ratio equal to the overall Si/Al molar ratio determined by X-ray fluorescence and greater than 0.044 and less than 17.

According to the invention, said catalyst used in the oligomerization method of the invention is preferably constituted entirely by said silica-alumina; it is devoid of any other element. The formed silica-alumina, having the characteristics in terms of pore volumes V1, V2 and advantageously V3 as defined previously in the present description, then constitutes the oligomerization catalyst.

However, the oligomerization catalyst used in the method according to the invention can advantageously contain a binder. For example, said binder is selected from the group comprising silica, alumina, clays, titanium dioxide, boron oxide and zirconia and any mixture of the binders previously mentioned. The preferred binders are silica and alumina and even more preferably alumina in all its forms known to a person skilled in the art, for example gamma alumina. The content by weight of binder in the catalyst is between 1 and 40% and even more preferably between 5% and 20%. According to the invention, the silica-alumina formed with a binder, preferably with a silica or an alumina, then constitutes the oligomerization catalyst and has the characteristics in terms of pore volumes V1, V2 and advantageously V3 as defined previously in the present description.

The NMR MAS spectra of the solid of ²⁷Al of the oligomerization catalyst used in the method of the invention show two separate multiplets of peaks. A first type of aluminium the maximum of which resonates around 10 ppm extends between −100 and 20 ppm. The position of the maximum suggests that these species are essentially of the Al_{VI} type (octahedral). A second type of minority aluminium the maximum of which resonates around 60 ppm extends between 20 and 110 ppm. This multiplet can be broken down into at least two species. The predominant species of this multiplet would correspond to the Al_{IV} atoms (tetrahedral). For the oligomerization catalyst used in the oligomerization method of the present invention, the proportion of the octahedral Al_{VI} is advantageously greater than 50 mol. %, preferably greater than 60 mol. % and even more preferably greater than 70 mol %.

The acidity of the oligomerization catalyst used in the method according to the invention is advantageously measured by IR monitoring of the thermal desorption of pyridine. Generally, the B/L ratio, as described previously in the present description, of the oligomerization catalyst used in the method according to the invention is comprised between 0.05 and 1, preferably between 0.05 and 0.7, and very preferably between 0.05 and 0.5.

The oligomerization catalyst used in the method of the invention can optionally contain at least one metallic element selected from the metals of groups IVB, VB, VIB and VIII and mixtures thereof. Among the metals of group IVB, titanium, zirconium and/or hafnium can be present in the catalyst. Among the metals of group VB, vanadium, niobium and/or tantalum can be present in the catalyst. Among the metals of group VIB, chromium, molybdenum and/or tungsten can be present in the catalyst. Among the metals of group VIII, the metals belonging to the first row of metals of group VIII, namely iron, cobalt and nickel, are preferred. The content of these metals can be up to 1.0 wt. % of the final catalyst. The catalyst can optionally also contain silicon as doping element deposited on the silica-alumina.

Preparation of the Catalyst Used in the Oligomerization Method According to the Invention

Any method for the synthesis of silica-alumina known to a person skilled in the art resulting in a silica-alumina that is homogeneous at the micrometre scale, and in which the cationic impurities can be lowered to less than 0.1 wt. % and the anionic impurities can be lowered to less than 1 wt. % and that has a pore distribution in terms of pore volumes V1, V2 and advantageously V3 as defined previously in the present description is suitable for preparing the oligomerization catalyst used in the method of the invention.

A preferred method of synthesis consists of mixing an aluminium compound with a compound of silicic acid in an aqueous medium, then drying and calcining the product obtained.

Preferably, aluminium alcoholates are used as alumina precursors and orthosilicic acid, advantageously purified on ion exchangers, as silica precursor. The method of preparation of the silica-alumina present in the oligomerization catalyst comprises hydrolysis of the aluminium alcoholate with water, advantageously purified on ion exchangers, and simultaneous or subsequent addition of orthosilicic acid, advantageously purified on ion exchangers. More preferably, the aluminium alcoholates are hydrolysed with water purified on ion exchangers, whereas orthosilicic acid purified on ion exchangers is preferably added simultaneously in a quantity from 0.1 to 15 wt. %, preferably from 0.1 to 10 wt. %, in hydrolysis water, or alternatively the alumina/water mixture obtained after hydrolysis with water purified on ion exchangers is mixed with solution of orthosilicic acid purified on ion exchangers at a concentration from 0.1 to 15 wt. %, preferably from 0.1 to 10 wt. %. The addition of orthosilicic acid to the alumina/water mixture can for example be carried out in a stirred reactor.

According to a particular embodiment of the method of preparation of the silica-alumina, a minor proportion of at least one stabilizing element selected from the group comprising zirconia and titania is added together with the solution of orthosilicic acid or after the addition of the latter. The stabilizing element is preferably added in the form of a soluble salt.

At the end of said preparation, the silica-alumina is obtained in the form of powder, which is then formed as described below. Said powder can be obtained by precipitation/gelation and filtration, by spraying of a suspension and by any other methods that are well known to a person skilled in the art.

Forming of the Oligomerization Catalyst

The oligomerization catalyst used in the method according to the invention is in the form of spheres, pellets or extrudates, preferably extrudates. Very advantageously, said oligomerization catalyst is in the form of extrudates with a diameter between 0.5 and 5 mm and more particularly between 0.7 and 2.5 mm. The shapes are cylindrical (which can be hollow or solid), cylindrical-twisted, multilobed (2, 3, 4 or 5 lobes for example). The extrudates can also be in the form of rings. The cylindrical and multilobed shapes are preferably used, but any other shape can be used.

The oligomerization catalyst used in the method of the invention is obtained by forming at least said silica-alumina present in said catalyst by any technique known to a person skilled in the art. Forming can be carried out for example by extrusion, coating, pelletization, drying, spraying, by the oil-drop method, by rotating plate granulation or by any other method well known to a person skilled in the art. Forming is carried out in the presence of the various constituents of the catalyst.

More precisely, when the catalyst is in the form of extrudates, water can be added or withdrawn in order to adjust the viscosity of the paste to be extruded. This stage can be performed at any point of the mixing stage of the silica-alumina. To adjust the solids content of the paste to be extruded in order to make it extrudable, it is also possible to add a compound that is predominantly solid and preferably an oxide or a hydrate. A hydrate will preferably be used, and even more preferably a hydrate of aluminium. The loss on ignition of this hydrate is greater than 15%.

Mixing is preferably carried out in the presence of an acid. The content of acid added during mixing prior to forming is less than 30 wt. %, preferably comprised between 0.5 and 20 wt. % of the anhydrous mass of silica-alumina used during synthesis. Extrusion can be carried out using any conventional, commercially available tool. The paste resulting from mixing is extruded through a die, for example using a piston or a single-screw or double-screw extruder. This extrusion stage can be carried out by any method known to a person skilled in the art. The extrudates of the oligomerization catalyst used in the method of the invention generally have a crushing strength of at least 70 N/cm and preferably greater than or equal to 100 N/cm. Preferably, the extrudates are constituted entirely by silica-alumina and have the characteristics in terms of pore volumes V1, V2 and advantageously V3 as defined previously in the present description.

Moreover, said oligomerization catalyst used in the method according to the present invention can have been treated as is well known to a person skilled in the art with additives for facilitating forming and/or improving the final mechanical properties of said catalyst based on silica-alumina. As examples of additives, in particular cellulose, carboxymethylcellulose, carboxyethylcellulose, xanthan gums, surfactants, flocculants such as polyacrylamides, carbon black, starches, stearic acid, polyacrylic alcohol, polyvinyl alcohol, biopolymers, glucose, polyethylene glycols, etc may be mentioned.

Control of the characteristic porosity of the oligomerization catalyst used in the method of the invention is achieved partially during this stage of forming of the particles of catalyst.

Drying and Calcining of the Oligomerization Catalyst

For the preparation of the oligomerization catalyst, one or more stages of drying and one or more stages of calcining are carried out during implementation of the method of preparation of the oligomerization catalyst.

Drying is carried out by any technique known to a person skilled in the art. Preferably, drying is carried out at a temperature comprised between 80 and 600° C., preferably between 300 and 600° C.

In order to obtain the catalyst used in the oligomerization method of the present invention, it is preferable to calcine in the presence of molecular oxygen, for example in an air stream, at a temperature less than or equal to 1100° C. At least one calcining can be carried out after any one of the preparation stages. This treatment can, for example, be carried out in a traversed bed, in a swept bed or in static atmosphere. For example, the furnace used can be a rotary furnace or can be a vertical furnace with radial traversed layers. The calcining conditions (temperature and time) depend mainly on the maximum usage temperature of the catalyst, the preferred calcining conditions being between more than one hour at 200° C. and less than one hour at 1100° C. Calcining can be carried out in the presence of steam. The final calcining can optionally be carried out in the presence of an acid or basic vapour. For example, calcining can be carried out under partial pressure of ammonia.

According to a very preferred embodiment of the method of preparation of the oligomerization catalyst, the first step is obtaining the solid constituted by at least said silica-alumina according to a method using aluminium alcoholates and orthosilicic acid as described previously in the present description. Then said solid is formed as extrudates in the manner described above. Said extrudates are then dried and calcined.

Post-Synthesis Treatments Applied on the Formed and Calcined Catalyst

Post-synthesis treatments can be carried out in order to improve the properties of the catalyst, in particular its homogeneity at the micrometre scale as defined previously.

According to a preferred embodiment, the post-synthesis treatment is a hydrothermal treatment. The hydrothermal treatment is carried out by any technique known to a person skilled in the art. Hydrothermal treatment means contacting the oligomerization catalyst with water in the vapour phase or in the liquid phase. Hydrothermal treatment can in particular comprise ripening, steaming, autoclaving, calcining under moist air, rehydration. Without reducing the scope of the invention, such a treatment has the effect of making the silica component mobile. Ripening takes place advantageously before or after forming.

According to a preferred implementation of the hydrothermal treatment, steaming is carried out in a furnace in the presence of steam. The temperature during steaming can be between 600 and 1100° C. and is preferably greater than 700° C. for a period of time between 30 minutes and 3 hours. The steam content is greater than 20 g of water per kg of dry air and preferably more than 40 g of water per kg of dry air and more preferably more than 100 g of water per kg of dry air. Said treatment can if necessary replace the calcining treatment completely or partly.

The catalyst can also be subjected advantageously to a hydrothermal treatment in confined atmosphere. By hydrothermal treatment in confined atmosphere is meant treatment of the catalyst by passage through an autoclave in the presence of water at a temperature greater than ambient temperature.

In the course of this hydrothermal treatment, the catalyst comprising said silica-alumina can be treated in various ways. Thus, the catalyst can be impregnated with an acid, basic or neutral solution, prior to its passage through the autoclave, autoclaving of the catalyst being carried out either in the vapour phase, or in the liquid phase, and said vapour or liquid phase of the autoclave can be acid, basic or neutral. This impregnation prior to autoclaving can be acid, basic or neutral. This impregnation prior to autoclaving can be carried out dry or by immersion of the catalyst in an acid, basic or neutral aqueous solution. By dry impregnation is meant contacting the catalyst with a volume of solution less than or equal to the total pore volume of the silica-alumina treated. Preferably, impregnation is carried out dry.

The autoclave is preferably a rotating basket autoclave as defined in patent application EP-A-0 387 109.

The temperature during autoclaving can be between 100 and 250° C. for a period of time between 30 minutes and 3 hours.

Oligomerization Method According to the Invention

The method according to the invention is a method of oligomerization of olefins for the production of fuel, for example the production of gasoline and/or kerosene and/or diesel fuel, from a light olefinic hydrocarbon feed containing between 2 and 10 carbon atoms per molecule, preferably between 2 and 8 carbon atoms per molecule, and in particular from a light olefinic hydrocarbon feed containing a high proportion of propylene and/or butenes and/or pentenes using an oligomerization catalyst comprising at least one silica-alumina, which has, after forming, the characteristics in terms of pore volumes V1, V2 and advantageously V3 as defined previously in the present description.

The olefinic hydrocarbon feed used in the oligomerization method according to the invention contains from 20 to 100 wt. %, and preferably from 25 to 80 wt. % of olefins. The olefins present in the olefinic hydrocarbon feed can be derived for example from a catalytic cracker and/or from a steam cracker and/or from a paraffin dehydrogenation unit and/or from a unit for polymerizing dehydration of methanol to water and light olefins and/or from any other sources leading to the production of light olefins.

Feed Used in the Oligomerization Method According to the Invention

The olefinic hydrocarbon feed sent to the oligomerization reactor used for implementing the oligomerization method of the invention, containing said catalyst based on at least said silica-alumina, is preferably cleaned of impurities, such as for example water, sulphur-containing derivatives, and basic nitrogenous derivatives, before being fed into the oligomerization reactor.

The olefinic hydrocarbon feed can be an olefinic C4 cut, which usually comprises, to more than 90 wt. %, isobutane, n-butane, 1-butene, 2-butenes, isobutene and optionally a small quantity of butadiene. The butadiene is generally removed upstream of the oligomerization by selective hydrogenation.

The olefinic hydrocarbon feed can also be an olefinic C3-C4 cut. The composition of the olefinic C3-C4 cut is very variable depending on its provenance. It can comprise between about 20 and 50 wt. % of propylene and propane, between about 50 and 80 wt. % of isobutane, n-butane, 1-butene, 2-butenes, isobutene and optionally a small quantity of butadiene. The butadiene is generally removed upstream of the oligomerization by selective hydrogenation.

The olefinic hydrocarbon feed can moreover be an olefinic C3 cut. It usually comprises at least 90 wt. % of propylene and propane.

The olefinic hydrocarbon feed can also be an olefinic C5 cut. The composition of the olefinic C5 cut is very variable depending on its provenance. It comprises advantageously between 30 and 80 wt. % of olefinic C5, between 1 and 20 wt. % of olefinic C6 and between I and 20 wt. % of olefinic C4.

The olefinic hydrocarbon feed can also be a cut containing olefins with more than four carbon atoms, designated C4+cut. The composition of the olefinic C4+cut is very variable depending on its provenance. It comprises advantageously between 30 and 80 wt. % of olefinic

According to the invention, the exothermic character of the oligomerization reaction can be managed by recycling at least a proportion of the unconverted effluent, which contains in particular the paraffins that were not converted during the reaction, to the oligomerization reactor and/or by dilution of the feed by adding paraffins originating from another source, said paraffins being of the same molecular weight and/or heavier than the olefinic feed, said paraffins being aliphatic or cyclic, and/or by recycling a proportion of the oligomers formed.

In all cases of methods leading to the formation of gasoline and/or kerosene and/or diesel fuel and/or more generally an olefinic cut with an initial boiling point at a temperature greater than 50° C., the olefinic cuts obtained leaving the process can optionally be hydrogenated, partially or completely.

Operating Conditions of the Oligomerization Method According to the Invention

Said oligomerization method is preferably used under the following operating conditions: total pressure comprised between 0.1 and 20 MPa and preferably between 0.2 and 7 MPa; temperature comprised between 30° C. and 600° C. and preferably between 40° C. and 400° C.; hourly space velocity (HSV) comprised between 0.01 and 100 h⁻¹ and preferably between 0.05 and 20 h⁻¹.

According to the invention, the oligomerization method corresponds to an addition limited essentially to 2 to 10 monomers or basic molecules, said monomers being olefins.

Embodiments of the Oligomerization Method of the Invention

### First Embodiment: Selective Oligomerization

According to said first embodiment, an olefinic C4 cut is contacted with the catalyst comprising said silica-alumina as described in the present invention so as to limit the overall conversion of the n-butenes to less than 10 wt. %, preferably to less than 5 wt. %, whereas more than 90 wt. % of the quantity of isobutene is converted, preferably more than 95 wt. %. The isobutene is converted at more than 90 wt. % to dimers and trimers. Then the oligomerization effluent is subjected to distillation in such a way that one of the fractions recovered (light effluent) contains more than 90 wt. % of butane, isobutane and the butenes that had not reacted during oligomerization, with at least a proportion of this fraction then feeding for example an alkylating unit or a hydration unit, whereas the other fraction constituted by the oligomers obtained is used as gasoline base, optionally after partial or total hydrogenation.

The embodiment of the oligornerization method described above corresponds to the embodiment called “selective oligomerization” in which the isobutene is predominantly converted.

According to said first embodiment of the oligomerization method of the invention, the oligomerization reaction is carried out at a temperature between 30° C. and 300° C., at a pressure between 0.1 and 20 MPa and the volume of olefinic hydrocarbon feed sent per volume of catalyst and per hour is between 0.05 and 5 h⁻¹. Preferably the temperature is between 40° C. and 160° C. and the pressure between 2 and 7 MPa, so as to ensure that the reaction takes place in the liquid phase, or at least in homogeneous phase (i.e. entirely in the liquid phase or entirely in gas phase), and the volume of olefinic hydrocarbon feed sent per volume of catalyst and per hour is preferably between 0.1 and 2.5 h⁻¹.

The technology of the oligomerization reactor can be a fixed bed, a fluidized bed or a circulating bed. The preferred technology is implementation in a fixed bed.

Preferably, the oligomers thus obtained are reinjected into a supplementary oligomerization reactor containing for example the oligomerization catalyst based on at least said silica-alumina as described previously, so as to increase the chain length of the oligomers and thus attain the kerosene cut and/or the diesel cut, or more generally an olefinic cut with an initial boiling point at a temperature at least greater than 150° C.

Advantageously, the light effluent from oligomerization, i.e. the C4 cut, can be fed into a hydroisomerization reactor for the purpose of hydroisomerization of a proportion of the unconverted 1-butene to 2-butene, so as to get closer to thermodynamic equilibrium. The other constituents of the effluent are not then converted significantly during the hydroisomerization stage. Conversion of 1-butene to 2-butene is very useful if the C4 fraction thus obtained at the outlet of the hydroisomerization reactor can then be fed into a reactor for aliphatic alkylation with hydrofluoric acid, the products obtained by alkylation of 2-butene with isobutane having a better octane number than the alkylate obtained from 1-butene.

In view of the highly exothermic character of the oligomerization reaction, the quantity of isobutene in the hydrocarbon feed to the oligomerization reactor is preferably less than 35 wt. %, even more preferably less than 15 wt. %, said quantity being optionally obtained by diluting the feed, for example with butane or isobutane or raffinate from the oligomerization unit.

### Second Embodiment

According to said second embodiment, an olefinic C4 cut or an olefinic C3-C4 cut is contacted with the oligomerization catalyst comprising at least said silica-alumina as described previously in the present invention so that a proportion of the butenes contained in the hydrocarbon feed is converted to dimers or timers, which are then used as gasoline base. In accordance with said second embodiment of the method of the invention, less than 80 wt. % of the butenes is converted and at least 80 wt. %, preferably at least 90 wt. % of the isobutene is converted. This method makes it possible to produce a maximum quantity of gasoline while minimizing the quantities of kerosene and diesel formed.

In the oligomerization reactor used for applying said second embodiment, the temperature is between 40° C. and 250° C., preferably between 50° C. and 200° C., and the pressure is between 0.1 and 10 MPa, preferably between 0.1 and 6 MPa, and the quantity of hydrocarbon feed sent per volume of catalyst and per hour is between 0.05 and 5 h⁻¹, preferably between 0.1 and 2.5 h⁻¹. The technology of the reactor can be a fixed bed, a fluidized bed or a circulating bed. The preferred technology uses a fixed bed.

A variant of said second embodiment of the method of the invention consists of using as feed, an olefinic feed from which isobutene has been removed beforehand either partly or completely, for example by using, upstream of the oligomerization unit, an etherification unit by selectively reacting the isobutene with an alcohol, for example methanol or ethanol, without converting the n-butene, or else by using, upstream of the oligomerization unit, a unit for selective oligomerization such as that described above in said first embodiment. The oligomers produced then have less branching than those obtained by treatment of the complete cut including isobutene.

### Third Embodiment

A third embodiment of the method according to the invention consists of subjecting an olefinic C4 cut, optionally containing traces of propylene, to oligomerization in such a way that the main proportion of the butenes contained in the feed is converted to dimers or trimers, which are then used as gasoline base. In accordance with said third embodiment of the method of the invention, at least 90 wt. % of the 1-butene, at least 80 wt. % of the 2-butenes, at least 97 wt. % of the isobutene and at least 80 wt. % of the propylene are converted. Said third embodiment of the method of the invention makes it possible to produce a maximum quantity of gasoline without producing kerosene or diesel.

The olefinic C4 cut usually comprises isobutane, n-butane, 1-butene, 2-butene, isobutene and optionally a small quantity of butadiene. The butadiene is generally removed upstream of oligomerization by selective hydrogenation, to avoid reactions of polymerization which would inactivate the catalyst.

Said method used according to said third embodiment comprises the following stages:
a first stage of oligomerization; an olefinic C4 cut is treated in a first oligomerization reactor in which the overall conversion of the n-butenes contained in the feed is less than 45 wt. % and the conversion of the isobutene is greater than 80 wt. %, preferably greater than 85 wt. %, the oligomers obtained being dimers and trimers to more than 80 wt. %,
the effluent from the first stage of oligomerization is sent to a fractionating column so as to recover a first fraction containing isobutene and unconverted n-butenes and a second fraction comprising 90 wt. % of dimers and trimers from the oligomerization reaction,
a second stage of oligomerization: said recovered first fraction is fed into a second oligomerization reactor in which the olefins are largely converted to dimers and trimers, i.e. at least 50 wt. % of the n-butenes are converted, preferably at least 75 wt. % of the 1-butene and at least 55 wt. % of the 2-butene are converted and,
the effluent from the second stage of ohgomerization is sent to the fractionating column associated with the first oligomerization reactor or to a second column for separating gasoline or kerosene or diesel from the unconverted C4 compounds.

In the oligomerization reactors, the temperature is between 40° C. and 250° C., preferably between 45° C. and 200° C., and the pressure is between 0.1 and 10 MPa, preferably between 0.1 and 6 MPa, and the quantity of hydrocarbon feed sent per volume of catalyst and per hour is between 0.05 and 5 h⁻¹, preferably between 0.1 and 2.5 h⁻¹. The technology of the reactor can be a fixed bed, a fluidized bed or a circulating bed. Preferably, the technology is a fixed bed.

Preferably, in the second oligomerization reactor, the operating conditions are harsher than in the first reactor.

Said third embodiment of the method of the invention can be applied to an olefinic C3-C4 feed.

### Fourth Embodiment

According to said fourth embodiment, an olefinic C4 cut or an olefinic C3-C4 cut is contacted with the oligomerization catalyst comprising said silica-alumina as described in the present invention in such a way that the major proportion of the butenes contained in the feed is converted, so as to form a gasoline base and a kerosene base. In accordance with said fourth embodiment of the method of the invention, at least 90 wt. % of the 1-butene, at least 80 wt. % of the 2-butenes and at least 97 wt. % of the isobutene are converted. The olefinic C4 cut usually comprises essentially isobutane, n-butane, 1-butene, 2-butene, isobutene and optionally a small quantity of butadiene. The olefinic C3-C4 cut further comprises propane and propylene in the proportions given above in the present description.

In the oligomerization reactor, the temperature is between 60° C. and 250° C., preferably between 100° C. and 200° C., and the pressure is between 0.1 and 10 MPa, preferably between 0.1 and 6 MPa, and the quantity of hydrocarbon feed sent per volume of catalyst and per hour is between 0.05 and 5 h⁻¹, preferably between 1 and 2.5 h⁻¹. The technology of the reactor can be a fixed bed, a fluidized bed or a circulating bed. Preferably the technology is a fixed bed.

### Fifth Embodiment

According to said fifth embodiment, an olefinic C3 cut is contacted with said oligomerization catalyst comprising said silica-alumina as described in the present invention in such a way that the major proportion of the propylene contained in the feed is converted, i.e. at least 80 wt. % of the propylene contained in the feed is converted, so as to form a gasoline base and a kerosene base. The olefinic C3 cut usually comprises at least 90 wt. % of propylene and propane.

The oligomerization reaction is carried out at a temperature between 30° C. and 300° C., at a pressure between about 0.1 and 20 MPa and the volume of hydrocarbon feed sent per volume of catalyst and per hour is between 0.05 and 5 h⁻¹. Preferably the temperature is between 40° C. and 160° C., the pressure is between 2 and 7 MPa, the volume of hydrocarbon feed sent per volume of catalyst and per hour is preferably between 0.1 and 2.5 h⁻¹. The technology of the reactor can be a fixed bed, a fluidized bed or a circulating bed. The preferred technology is implementation in a fixed bed.

### Sixth Embodiment

According to said sixth embodiment, an olefinic cut containing olefins with more than four carbon atoms, for example a cut originating from an FCC (fluidized-bed catalytic cracking) process, is contacted with said oligomerization catalyst comprising said silica-alumina as described in the present invention in such a way that the major proportion of the olefins containing at least 4 carbon atoms contained in the feed is converted, i.e. at least. 70 wt. % of the olefins contained in the feed is converted, so as to form a gasoline base, a kerosene base or a diesel base.

The oligomerization reaction is carried out at a temperature between 30° C. and 300° C., at a pressure between about 0.1 and 20 MPa and the volume of hydrocarbon feed sent per volume of catalyst and per hour is between 0.05 and 5 h⁻¹. Preferably the temperature is between 40° C. and 160° C., and the pressure between 2 and 7 MPa, and the volume of hydrocarbon feed sent per volume of catalyst and per hour is preferably between 0.1 and 2.5 h⁻¹. The technology of the reactor can be a fixed bed, a fluidized bed or a circulating bed. The preferred technology is implementation in a fixed bed. The method of conversion using a recycling schema can also be used.

The examples given below illustrate the present invention without limiting its scope.

### EXAMPLE 1

### Preparation of a Catalyst C1 constituted by a Silica-Alumina SA1 (according to theinvention)

Catalyst C1 is prepared by extrusion of a silica-alumina SA1 without binder. In this example, catalyst C1 is therefore identical to the formed silica-alumina SA1. Catalyst C1 has a chemical composition by weight of 71% Al₂O₃ and 29% SiO₂.

45.0 g of aluminium hexanolate is hydrolysed continuously using 45.5 g of demineralized water in a stirred reactor, at 90° C. for 45 minutes. Then the aqueous suspension of alumina that separates from the alcohols is mixed with orthosilicic acid that has been demineralized on an ion exchanger in a total quantity of 8.9 wt. %. Then the suspension obtained is dried conventionally using a sprayer at a temperature from 300° C. to 600° C. and the silica-alumina SA1 is thus obtained. The powder thus prepared is formed in a Z-blade mixer in the presence of 3 wt. % of nitric acid relative to the anhydrous product. Extrusion is carried out by passing the paste through a die with orifices of diameter equal to 2.5 mm. The extrudates of catalyst C1 thus obtained are dried at 150° C., and then calcined at 550° C. The Si/Al ratios measured by XRF and TEM analyses coupled with EDX of catalyst C1 are 0.35 and 0.33 respectively. The sodium and chlorine contents of catalyst C1 are 0.01% and 0.02 wt. % respectively. The extrudates are cylindrical with diameter equal to 1.6 mm. The specific surface of catalyst C1 is 307 m²/g. The total pore volume of catalyst C1, measured by, the nitrogen adsorption isotherm, is equal to 0.59 ml/g. Its total pore volume, measured by mercury porosimetry, is equal to 0.78 ml/g. The average diameter of the pores of catalyst C1 determined by mercury porosimetry is 7.6 nm. The volume V1 of mesopores with a diameter comprised between 4 and 15 nm is 0.40 ml/g and this volume represents about 51% of the total pore volume. The. volume V2 of macropores of the catalyst, with diameter greater than 50 nm, is 0.32 cm³/g and represents about 41% of the total pore volume. The volume V3 of the pores of the catalyst with diameter greater than 25 nm is 0.35 ml/g and represents about 45% of the total pore volume. The TD of the catalyst is 0.50 g/cm³. The B/L ratio has a value of the order of 0.1.

### EXAMPLE 2

### Preparation and forming of a Catalyst C2 constituted by a Silica-Alumina SA2 (notaccording to the invention)

Catalyst C2 is prepared by extrusion of a silica-alumina SA2 with boehmite. The chemical composition by weight of catalyst C2 is 71% Al₂O₃ and 29% Si0₂.

An aqueous solution of aluminium sulphate (6.1 wt. % aluminium) and an aqueous solution based on sodium silicate (10.2 wt. % silicon) and sodium hydroxide (9.7 wt. %), maintained at a temperature of 30° C., are fed into a reactor containing 1.2 1 of demineralized water heated to 30° C. The pH of the entire solution during addition to the reactor is maintained at 8 using a sulphuric acid solution. The suspension thus obtained is filtered. The resultant silica-alumina SA2 has a chemical composition by weight of 30% Al₂O₃ and 70% SiO₂. The cake from filtration of SA2 is recovered and then mixed in a Z-blade mixer in the presence of 3 wt. % of nitric acid, relative to the anhydrous product, and boehmite so as to reach the chemical composition by weight of catalyst C2 of 71% Al₂O₃ and 29% SiO₂. Extrusion is carried out by passing the paste through a die with orifices of diameter equal to 2.5 mm. The extrudates of catalyst C2 thus obtained are dried at 150° C., and then calcined at 550° C.

The specific surface of catalyst C2 is 260 m²/g. Its total pore volume, measured by the nitrogen adsorption isotherm, is equal to 0.57 ml/g. Its total pore volume measured by mercury porosimetry is equal to 0.44 ml/g. The average diameter of the pores of catalyst C2 determined by mercury porosimetry is 7.7 nm. The volume V1 of mesopores (diameter between 4 and 15 nm) of catalyst C2 is 0.40 ml/g and this volume represents about 83% of the total pore volume. The volume V2 of macropores (diameter greater than 50 nm) of catalyst C2 is 0.021 ml/g and represents less than about 5% of the total pore volume. The volume V3 of the pores (diameter greater than 25 nm) of catalyst C2 is 0.07 ml/g and represents less than 16% of the total pore volume. The TD of the catalyst is 0.61 g/cm³.

### EXAMPLE 3

### Catalytic Evaluation of Catalysts C1 and C2 in method of oligomerization of lightolefins (First Embodiment)

An olefinic C4 cut originating from a catalytic cracker is dried on a molecular sieve of the 13X type to remove traces of sulphur and water. The composition of the feed at the end of these treatments is presented in Table 1.

TABLE 1
composition of the feed for oligomerization of light olefins.

| Compounds | Content (wt. %) |
|---|---|
| isobutane | 30.7 |
| n-butane | 12.7 |
| isobutene | 15.6 |
| 1-butene | 9.4 |
| 2-butene | 31.6 |

Catalysts C1 and C2 are loaded in a fixed-bed reactor and tested for the oligomerization reaction of the feed described in Table 1. The operating conditions used are listed in Table 2.

The catalysts are activated in situ before the oligomerization reaction under N₂ at 250° C. for 4 hours.

TABLE 2
Operating conditions of the oligomerization reaction.

| | |
|---|---|
| Pressure range | 25-65 MPa |
| Temperature range | 60° C.-120° C. |
| HSV | 0.85 h⁻¹ |

HSV (h⁻¹) = [volume of catalyst/volume flow rate of feed]: 0.85 h⁻¹

The performance of the catalysts is evaluated in relation to the selectivity for required product for conversions to equivalent olefins. The selectivity of the 155⁻ cut is defined as the ratio of the mass of products having a boiling point less than 155° C. to the total mass of reaction products. The 155⁻ cut, which comprises products having a boiling point less than 155° C., is a gasoline fraction. Similarly, the selectivity of the 155-225 cut is defined as the fraction by weight of products having a boiling point in the range 155° C.-225° C. The 155-225 cut, which comprises products having a boiling point in the range 155° C. 225° C., is a kerosene cut. The selectivities by mass for the 155⁻ and 155-225 cuts of catalysts C1 and C2 under the operating conditions of the test are summarized in Table 3.

TABLE 3
performance of catalysts C1 and C2.

| Catalyst | Catalyst C1 | | Catalyst C2 | |
|---|---|---|---|---|
| Reaction temperature | 60° C. | 110° C. | 60° C. | 110° C. |
| Selectivity of the 155⁻ cut | 62.0 | 56.5 | 60.0 | 54.0 |
| Selectivity of the 155-225 cut | 32.2 | 33.3 | 33.7 | 34.6 |
| Selectivity of the 225⁻ cut | 94.2 | 89.8 | 93.7 | 88.6 |
| Selectivity of the 225⁺ cut | 5.8 | 10.2 | 6.3 | 11.4 |

In iso-conversion to olefins, catalyst C1 formed from a macroporous silica-alumina after forming is more selective than catalyst C2 comprising a non-macroporous silica-alumina after framing: catalyst C1 promotes the production of oligomers having a boiling point less than 225° C., at the expense of the production of heavier products having a boiling point greater than 225° C.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding FR application No. 09/04.818, filed Oct. 8, 2009, are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. Method A method of oligomerization of an olefinic hydrocarbon feed comprising contacting said feed with at least one catalyst comprising at least one silica-alumina, the silica content by weight of said catalyst being between 5 and 95 wt. % and the porosity of said silica-alumina when formed being such that:
i) the volume V1 of mesopores having a diameter comprised between 4 and 15 nm represents 30-80% of the total pore volume measured with a mercury intrusion porosimeter,
ii) the volume V2 of macropores having a diameter greater than 50 nm represents from 15 to 80% of the total pore volume measured with a mercury intrusion porosimeter.

2. A method of oligomerization according to claim 1, characterized in that said catalyst has a silica content by weight between 25 and 40 wt. %.

3. A method of oligomerization according to claim 1, characterized in that said silica-alumina when formed has a pore distribution such that said volume V2 of macropores represents from 35 to 80% of the total pore volume measured with a mercury intrusion porosimeter.

4. A method of oligomerization according to claim 1, characterized in that the average diameter of the pores of the formed silica-alumina, obtained using the mercury intrusion porosimeter, is in a range from 2 to 15 nm.

5. A method of oligomerization according to claim 1, characterized in that said silica-alumina when formed has a volume V3 of pores having a diameter greater than 25 nm between 20 and 80% of the total pore volume measured with a mercury intrusion porosimeter.

6. A method of oligomerization according to claim 1, characterized in that said silica-alumina when formed has a BET specific surface between 100 and 550 m²/g.

7. A method of oligomerization according to claim 1, characterized in that said silica-alumina when formed is a silica-alumina that is homogeneous at the micrometre scale.

8. A method of oligomerization according to claim 1, characterized in that said catalyst is constituted entirely by said silica-alumina.

9. Method A method of oligomerization according to claim 1, characterized in that said catalyst contains a binder.

10. A method of oligomerization according to claim 1, characterized in that said olefinic hydrocarbon feed contains from 25 to 80 wt. % of olefins.

11. A method of oligomerization according to claim 1, characterized in that said olefinic hydrocarbon feed is an olefinic C3 cut comprising at least 90 wt. % of propylene and propane.

12. A method of oligomerization according to claim 1, characterized in that said olefinic hydrocarbon feed is an olefinic C3-C4 cut.

13. A method of oligomerization according to claim 1, characterized in that said olefinic hydrocarbon feed is an olefinic C4 cut comprising, to more than 90 wt. %, isobutane, n-butane, 1-butene, 2-butenes, isobutene.

14. A method of oligomerization according to claim 1, characterized in that said olefinic hydrocarbon feed is an olefinic C5 cut.

15. A method according to claim 1 wherein the silica content is between 25 and 40 wt. %.

16. A method according to claim 1 having an Si/Al molar ration of between 0.28 and 0.56.

17. A method according to claim 5 wherein said volume V3 is between 30 and 50% of the toal pore volume.

18. A method according to claim 4 wherein said average diameter is in the range of 5 nm to 12 nm.

19. A method according to claim 15 having an Si/Al molar ration of between 0.28 and 0.56.

20. A method according to claim 6 wherein the specific surface is between 170 and 330 m²/g.
